(19) United States
(12) Patent Application Publication
(10) Pub. No.: US 2010/0143456 A1
(43) Pub. Date: Jun. 10, 2010

(54) MODEL MEMBRANE SYSTEMS

(76) Inventors: Joseph Altin, Holder (AU); Christopher Richard Parish, Campbell (AU)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/638,373

(22) Filed: Dec. 15, 2009

Related U.S. Application Data
(63) Continuation of application No. 11/688,621, filed on Mar. 20, 2007, now abandoned.
Application No. 11/688,621 is a continuation of application No. 10/031,859, filed on Feb. 26, 2002, now abandoned.
Application No. 10/031,859 is a continuation of application No. PCT/AU00/00397, filed on Apr. 28, 2000.

(30) Foreign Application Priority Data
Apr. 28, 1999 (AU) AU PQ 0023

(51) Int. Cl. A61K 9/127; A61K 35/00; A61K 39/00; A61K 35/12; A61P 43/00

(52) U.S. Cl. 424/450; 424/93.1; 424/184.1; 424/93.7

(57) ABSTRACT

The present invention relates generally to modifying biological and/or synthetic membranes or liposomes, or combinations thereof, for the purpose of altering immunity, or for the targeting of drugs and other agents to a specific cell type or tissue when administered in vivo to achieve a therapeutic effect. Modification of the membranes is achieved by incorporation and/or attachment of metal chelating groups, thereby allowing engraftment of one or more targeting molecules possessing a metal affinity tag, and a targeting of the engrafted membranes to specific cell types or tissues in vivo. The invention, thus, provides a means of modifying the properties of biological and/or synthetic membranes and liposomes for the purpose of altering or enhancing immunity when used as vaccines, or for the targeting of encapsulated/incorporated drugs or other agents to a specific cell type or tissue when administered in vivo, to achieve a therapeutic effect or response, or for modifying a physiological response or biological function.

### CROSS REFERENCE TO RELATED APPLICATIONS

This application is a continuation of U.S. patent application Ser. No. 11/688,621, filed Mar. 2, 2007, which is a continuation of Ser. No. 10/031,859, filed Apr. 26, 2002, entitled “Model Membrane Systems”, which is a 371 of PCT/AUO/00397, filed Apr. 28, 2000, which claims priority to Australia Patent Application No. PQ0023, filed Apr. 28, 1999, subject matter of which all applications are incorporated

### FIELD OF THE INVENTION

The present invention relates generally to the use of metal chelator lipids to modify biological and/or synthetic membranes or liposomes for the purpose of altering biological responses, or for targeting these structures to a specific cell type or tissue to achieve a therapeutic effect, when administered in vivo. The invention provides a means of modifying the properties of biological and/or synthetic membranes and liposomes for the purpose of altering immunity when used as vaccines, or for the targeting of drugs and other agents to specific cell types or tissues when administered in vivo for therapeutic purposes or for modifying a physiological response or biological function.

### BACKGROUND OF THE INVENTION

Bibliographic details of the publications numerically referred to in this specification are collected at the end of the description.

In biological systems such as cells, bacteria or viruses, the surface membrane-associated biomolecules or receptors often exist as molecular structure consisting of two or more molecular components called subunits; these subunits may be identical, or molecularly distinct. The binding of natural ligand molecule(s) to receptor subunits may induce non-covalent aggregation or oligomerization of these receptor components on the lipid membrane structure. The oligomerization event is often an essential part of the mechanism by which the receptor can transduce transmembrane signals for triggering the induction of biological responses by the ligand molecule(s). In addition, the ability of certain receptors, or components thereof, to aggregate spontaneously may affect their ability to interact with ligands. Ligand molecules may be growth factors, cytokines, hormones, proteins, glycoproteins, polysaccharides, or any surface exposed or sub-cellular component of a cell, viral or subviral particle, or other infectious agent, which can bind to the receptor.

Recently a technique has been described in which the linkage of a recombinant hexa-histidine-tagged protein with nitrilotriacetic acid (NTA) is used to reversibly immobilize hex a-histidine-tagged proteins onto the solid sensing surface of a BIAcore surface plasmon resonance biosensor (1-5). The formation of a hybrid octadecanethiol/phospholipid membrane on the BIAcore sensing surface also has been described (6), enabling analysis of the binding of streptavidin to biotinylated phosphatidylethanolamine in the bilayer. In addition, the immobilization of histidine-tagged biomolecules to bilayer membranes via chelator lipids like NTA-dioctadecylamine has been demonstrated by epifluorescence microscopy and film balance techniques (7-8).

These prior art techniques do not describe a means of modifying the properties of biological and/or synthetic membranes and liposomes for the purpose of altering immunity when used as vaccines, or for the targeting of drugs and other agents to specific cells or tissues when administered in vivo for either therapeutic purposes or for modifying a physiological response or function.

There is a need, therefore, to anchor molecules to membranous materials for use, for example, in vaccine preparations, as agents for modifying immunological responses, as therapeutic agents, and for targeting drug delivery systems.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides a method of modifying the properties of biological and/or synthetic membranes or liposomes, or combinations thereof, for the purpose of altering immunity, or for the targeting of drugs and other agents to a specific cell type or tissue when administered in vivo for therapeutic purposes. The method comprises the use of some amphiphilic molecules which become incorporated into the said membrane or liposomes, wherein a proportion of the amphiphilic molecules have been modified by a covalent attachment of a metal chelating group such that some of the metal chelating groups are oriented toward the outside surface of said membrane; which method comprises the step of interacting a receptor domain which is covalently attached to a polypeptide tag with said membrane or liposome for a time and under conditions sufficient for said polypeptide tag to attach to said membrane via the outwardly facing metal chelating residues of said membrane, such that the receptor domains are capable of interacting specifically with a ligand molecule on cells and tissues within the body. Preferably, the specific interaction between the receptor domains and associated said membrane providing a means of altering immunity when used as vaccines, or of targeting membrane encapsulated or incorporated drugs and other agents to a specific cell type or tissue, when administered in vivo for therapeutic purposes or for modifying a physiological response or biological function.

One aspect of the present invention provides a method of modifying biological and/or synthetic membranes or liposomes, or combinations thereof, for the purpose of altering immunity, or for the targeting of drugs and other agents to a specific cell type or tissue when administered in vivo to achieve a specific therapeutic effect, said method comprising incorporating amphiphilic molecules into the said membrane or liposomes, wherein a proportion of the amphiphilic molecules have been modified by a covalent attachment of a metal chelating group such that at least some of the metal chelating groups are oriented toward the outside surface of said membrane or liposomes, which method also comprises the step of interacting a receptor domain which is covalently attached to a polypeptide tag with said membrane or liposomes for a time and under conditions sufficient for said polypeptide tag to attach to said membrane or liposomes via the outwardly facing metal chelating residues of said membrane or liposomes, such that the receptor domains are capable of interacting specifically with a ligand molecule that exists on a particular cell type or tissue within the body.

Another aspect of the present invention provides a method of targeting synthetic liposomes, made to encapsulate/incorporate a drug or therapeutic agent, to a specific cell type or tissue in vivo, by engrafting specific targeting molecules onto the liposomes, said method comprising:
(i) preparing a suspension of liposomes with chelator lipid incorporated, from a first lipid or phospholipid and a second lipid or phospholipid, wherein said second lipid or phospholipid has been modified by covalent attachment of a metal chelating group such as nitrilotriacetic acid (NTA), with some of .the NTA residues attached to the second lipid or phospholipid of said micelle (e.g. liposome) suspension oriented toward the outside surface of said membrane; such liposomes also can be prepared in the presence of, or be made to contain after preparation, any appropriate drug or therapeutic agent which can be encapsulated/incorporated into the liposomes;
(ii) incubating the liposomes with .a recombinant protein or target molecule bearing an appropriate metal affinity tag, for a time and under conditions sufficient for said polypeptide tag to attach via the NTA-chelating linkage to the outwardly-facing NTA residues of said liposomes;
(iii) if necessary, removing excess protein by washing, filtering or other washing means and suspending the liposomes in an appropriate solution; and
(iv) administering in vivo the engrafted liposomes containing the encapsulated/incorporated drug or agent to allow targeted delivery to a specific cell type or tissue to achieve a therapeutic effect.

A further aspect of the present invention contemplates a method for altering the immunogenicity of a target cell or membranous component thereof, said method comprising engrafting a molecule onto the membrane of said target cell or component by:
(i) preparing a suspension of chelator lipid or liposomes containing the chelator lipid;
(ii) incubating a suspension of cells or membranous structures with a suspension of the chelator lipid, or liposomes containing the chelator lipid, to allow the chelator lipid to incorporate into the membrane of cells or membranous components;
(iii) if necessary, washing away excess or unincorporated lipid or liposomes;
(iv) incubating the cells or membranous structures with a solution of said molecule to be anchored; and
(v) if necessary, washing away excess or unbound soluble molecule, and suspending the cells or structures in a solution suitable for administration in vivo.

Yet another aspect of the present invention provides a method of modifying biological and/or synthetic membranes and liposomes to achieve a specific therapeutic effect, such as the induction or modulation of an immune response or other physiological or biological response, when administered in vivo, said method comprising:-
(i) preparing a suspension of chelator lipid or liposomes composed of a mixture of lipids and the chelator lipid; or
(ii) incorporating the chelator lipid onto the cells or membranes, by incubating a suspension of the cells or membranes with a suspension of liposomes containing the chelator lipid, and if necessary, washing away excess or unincorporated lipid or liposomes;

(iii) incubating the liposomes, cells or membranous structures with a solution of a recombinant protein(s) or target molecule(s) possessing an appropriate metal affinity tag; and
(iv) washing away excess or unbound soluble protein, and suspending the liposomes, cells, or membranous structures in a solution suitable for administration in vivo.

Still another aspect of the present invention contemplates a method of targeting cells biological and/or synthetic membranes and liposomes to a particular cell type or tissue within the body, said method comprising engrafting onto the membrane structure a molecule having a binding partner on the particular cell or tissue to be targeted by:
(i) preparing a suspension of chelator lipid or liposomes containing the chelator lipid; or
(ii) incubating a suspension of the cells or membranous structures with a suspension of the chelator lipid to allow the lipid to become incorporated;
(iii) if necessary, washing away excess or unincorporated lipid;
(iv) incubating the liposomes or membranous structures with a solution of molecules to be anchored; and
(v) if necessary, washing away excess or unbound soluble molecule, and suspending the liposomes or structures in a solution suitable for administration in vivo.

Even yet another aspect of the present invention contemplates a method of treatment, said method comprising administering to a subject an effective amount of a liposome preparation or membranous material comprising an encapsulated or incorporated drug or active material, and an engrafted targeting molecule having a binding partner on the particular cell type or tissue to be targeted in vivo.

Even still another aspect of the present invention provides a vaccine composition comprising cells, liposomes, vesicles or membranous material having engrafted thereto molecules capable of modifying an immunological response to a subject to which the vaccine is administered, said vaccine further comprising one or more pharmaceutical carriers and/or diluents.

### BRIEF DESCRIPTION OF THE DRAWINGS

FIG. 1 is an illustration of the use of a metal-chelating linkage to modify the surface of cells, biological and/or synthetic membranous structures to alter the properties of these structures, thereby allowing them to be used for therapeutic purposes. The illustration depicts the engraftment of receptors bearing a hexa-histidine tag onto the surface of a cell or biological/synthetic membrane, such as the plasma membrane of cells or the membrane of sub-cellular membranous structures and onto the surface of artificial vesicles or liposomes. The recombinant protein is engrafted onto the membrane structure through the binding of the hexa-histidine tag on the protein to the NTA metal-chelating headgroup on the chelator lipid (denoted NTA-DTDA; this may also be referred to as di-C14-NTA) which has been incorporated into the membrane.

FIG. 2 is the fluorescence profile, as measured by fluorescence-activated cell sorting, of p815 cells engrafted with biotinylated and hexa-histidine-tagged CD40 and B7.1 molecules and then stained with streptavidin-FITC.

FIG. 3 is a graphical representation showing an induction of tumor-specific cytoxicity in T lymphocytes isolated from mice vaccinated with tumor cells bearing engrafted co-stimulator molecules. Syngeneic DBA/2 mice were immunized subcutaneously with either PBS or 1×10⁵ γ-irradiated P815 cells engrafted with the recombinant proteins: EPOR-6H, B7.1-6H, and B7.1-6H plus CD40-6H, as indicated. Spleens were removed from the mice 14 days after immunization, and T lymphocytes (effector T cells) were isolated, suspended in incubation medium and aliquoted into 24-well flat-bottom plates at a concentration of 1×10⁵ cells/well, and then co-cultured with 1×10⁵ γ-irradiated native P815 cells. After 5 days co-culture at 37° C. in the presence of 5% CO₂, the cells were incubated with ⁵¹Cr-labelled native P815 cell targets for 6 hrs at 37° C. at the indicated E:T ratio, before harvesting the supernatants and determining the amount of ⁵¹Cr released through specific lysis. Results are expressed as the percentage specific lysis±SEM, calculated as described in the Materials and Methods.

FIGS. 4(a) and (b) are graphical representations showing induction of tumor immunity by immunization with P815 tumor cells engrafted with recombinant co-stimulatory molecules. Mice were immunized by injection of either PBS or 1×10⁵ γ-irradiated P815 cells engrafted with recombinant protein(s) including: EPOR-6H, B7.1-6H, and B7.1-6H plus CD40-6H, as indicated. Two weeks after injection the mice in each group were challenged with 1×10⁵ native P815 cells by subcutaneous injection, and then monitored for tumor growth and survival. Each point in (A) represents the mean tumor diameter for each group of mice as a function of time for the first five weeks. The data in (B) show the percentage survival of the animals with time.

FIG. 5 is a graphical representation showing that binding of fluorescently-labelled liposomes to D10 cells (murine CD4+ T cells) is significantly greater when the liposomes are engrafted with either of the co-stimulatory molecules CD40 and B7.1, than when engrafted with a control protein EPOR. (Note that the D10 cells express ligands for B7.1 and CD40 but no ligand for EPOR.) The liposomes, composed of the lipids: PC:NTA-DTDA:FITC-PE (10:1:0.1 molar ratio), were engrafted with one or more recombinant protein (as indicated, EPOR, B7.1 and CD40) each bearing a hexa-histidine tag. The fluorescence profile of the cells in each condition (which reflects the extent of binding of the liposomes to the cells) was determined by fluorescence-activated cell sorting; the background fluorescence of cells (indicated “cells”) is shown for comparison. The results indicate that the binding of liposomes engrafted with an appropriate recombinant protein is specific for the type of engrafted protein and, therefore, that liposomes bearing engrafted recombinant proteins can be targeted to cells expressing the appropriate cognate receptor.

FIG. 6 is a graphical representation showing that synthetic liposomes engrafted with the co-stimulatory molecules B7.1 and CD40 can specifically stimulate the adherence of D10 cells (murine CD4+ T cells) to the culture dish. Cultured D10 cells were suspended in complete growth medium (RPMI 1640 plus 10% v/v FCS, 50 U/ml IL-2, antibiotics and 50 μM β-mercaptoethanol). The recombinant proteins EPOR, CD40 and B7.1 (each with a hexa-histidine tag) were mixed with the cells either in soluble form (as indicated sEPOR, sB7.1 and sCD40) or engrafted onto liposomes composed of PC and NTA-DTDA (10:1) (as indicated NTA-DTDA-EPOR, NTA-DTDA-B7.1 and NTA-DTDA-CD40), before plating the cells into separate wells of a 12-well Linbro tissue culture plate and incubating in growth medium for 2 hrs at 37° C. After the incubation, the non-adherent cells were removed from the wells by washing three times with PBS, and the remaining adherent cells were removed with a solution of 1 mM EDTA and counted microscopically. The data shows the proportion of adherent cells for each condition. The results demonstrate that liposomes bearing engrafted co-stimulatory molecules (i.e. B7.1 and/or CD40) can be used to modify immunological responses.

### DETAILED DESCRIPTION OF THE PREFERRED EMBODIMENTS

In one embodiment, the present invention contemplates the use of a method of engrafting the extramembranous or transmembrane domains of receptors onto biological and/or synthetic membranes or liposomes to overcome one or more of the foregoing shortcomings of the prior art.

Accordingly, the present invention provides a method of modifying biological and/or synthetic membranes or liposomes, or combinations thereof, for the purpose of altering immunity, or for the targeting of drugs and other agents to a specific cell type or tissue when administered in vivo to achieve a specific therapeutic effect, said method comprising incorporating amphiphilic molecules into the said membrane or liposomes, wherein a proportion of the amphiphilic molecules have been modified by a covalent attachment of a metal chelating group such that at least some of the metal chelating groups are oriented toward the outside surface of said membrane or liposomes, which method also comprises the step of interacting a receptor domain which is covalently attached to a polypeptide tag with said membrane or liposomes for a time and under conditions sufficient for said polypeptide tag to attach to said membrane or liposomes via the outwardly facing metal chelating residues of said membrane or liposomes, such that the receptor domains are capable of interacting specifically with a ligand molecule that exists on a particular cell type or tissue within the body.

Receptor domains may also be composed of proteins, glycoproteins or proteoglycans, oligosaccharides, or fragments or functional equivalents thereof.

A preferred metal chelating group for use in the present invention is nitrilotriacetic acid (NTA).

A biological membrane represents any membranous or lipid-containing material obtained from biological systems such as cells, tissues, bacteria, viruses, or components thereof.

Synthetic membranes and/or liposomes may be any artifitial lipid-containing structures such as a suspension of micelles (e.g. liposomes) formed from amphiphilic molecules, wherein a proportion of the amphiphilic molecules has been modified by covalent attachment of a metal chelating group. The synthetic membranes or liposomes can be formed by mechanical agitation of the lipid mixture in water or aqueous buffer such as by sonication and/or by the use of extrusion/filtration techniques and/or by the addition of water or aqueous buffers to an appropriate solution of amphiphilic molecules in an organic solvent or any combination of these.

The amphiphilic molecules are normally surfactant molecules having a hydrophilic “head” portion and one or more hydrophobic “tails”. Surfactants may be any of the known types, i.e. cationic (e.g. quaternary ammonium salts), anionic (e.g. organosulfonate salts), zwitterionic (e.g. the phospholipids: phosphatidylcholines and phosphatidylethanolamines), membrane spanning lipid, or non-ionic (e.g. polyether materials).

Synthetic membranes and/or liposomes may be comprised of more than one type of amphiphilic molecule. In a preferred embodiment, the synthetic membrane or liposome is comprised of a first phospholipid and a second phospholipid.

Thus, in a preferred form, the present invention contemplates a method of modifying biological and/or synthetic membranes and liposomes, by using metal chelating groups to engraft extramembranous or transmembrane receptor domains onto the said membranes or liposomes, thereby allowing these structures when administered in vivo to be targeted to any cell type or tissue that expresses a ligand for the receptor domains, for the purpose of achieving a therapeutic effect or for inducing or modifying a physiological response.

In a further preferred form, the first phospholipid is phosphatidylcholine (PC) and the second lipid is either nitrilotriacetic acid ditetradecylamine (NTA-DTDA) or nitrilotriacetic acid phosphatidylethanolamine (PE-NTA), and the molar ratio of PC:NTA-DTDA or PC:PE-NTA is about 10:1. However, the first phospholipid can be any phospholipid or hydrocarbon, or a mixture of any phospholipids or hydrocarbons, capable of forming a liposomal suspension; and the second phospholipid can be any lipid with a metal chelating headgroup which can be used to anchor or engraft receptor domains using a suitably engineered tag on the domain. In addition, the ratio of the first to the second phospholipid can be varied depending on the desired density of receptor domain molecules to be achieved on the biological membranes or liposomes.

Preferably, the polypeptide tag comprises a sequence of at least six amino acid residues such as a hexa-histidine molecule, but can be any sequence of amino acids that can bind strongly through the formation of a complex with the metal chelating component of a lipid containing a metal chelating group such as NTA. In one application of the subject invention the molecule is represented by the T cell costimulatory molecules B7.1 (CD80) and CD40. In another form of the instant invention, the molecule is the ligand called vascular endothelial cell growth factor (VEGF). More particularly, the receptor may be any cell surface receptor or ligand, or domains of such receptors or ligands.

Receptor domains can be engineered to have a hexa-histidine COOH-tail, or NH₂-tail using standard recombinant DNA techniques. A hexa-histidine tag also may be covalently attached to receptor domains, proteins, glycoproteins, polysaccharides, and other molecules by chemical means.

The present invention thus utilises metal chelating lipids to modify the properties of biological and/or synthetic membranes and liposomes, for therapeutic purposes and biological targeting in vivo to achieve a therapeutic effect. This technology is ideal in a preferred embodiment for modifying the properties of biological and/or synthetic membranes and liposomes for the purpose of altering immunity when used as vaccines, or for the targeting of drugs and other agents to specific cells or tissues when administered in vivo, for either therapeutic purposes, or for modifying a physiological response or biological function.

The use of a metal chelating linkage for modifying the biological properties of the said membrane systems of the present invention is also useful for targeting liposomes or vesicles which through the specificity of the liposome engrafted molecule(s) can target and deliver drugs, DNA/RNA or any therapeutic agent that can be encapsulated or incorporated into the liposomes, to specific cell types or tissues when the liposomes are administered in vivo.

According to this aspect of the present invention, there is provided a method of targeting synthetic liposomes, made to encapsulate/incorporate a drug or therapeutic agent, to a specific cell type or tissue in vivo, by engrafting specific targeting molecules onto the liposomes, said method comprising:-
(i) preparing a suspension of liposomes with chelator lipid incorporated, from a first lipid or phospholipid and a second lipid or phospholipid, wherein said second lipid or phospholipid has been modified by covalent attachment of a metal chelating group such as nitrilotriacetic acid (NTA), with some of the NTA residues attached to the second lipid or phospholipid of said micelle (e.g. liposome) suspension oriented toward the outside surface of said membrane; such liposomes also can be prepared in the presence of, or be made to contain after preparation, any drug or therapeutic agent which can be encapsulated/incorporated into the liposomes;
(ii) incubating the liposomes with a recombinant protein or target molecule bearing an appropriate metal affinity tag, for a time and under conditions sufficient for said polypeptide tag to attach via the NTA-chelating linkage to the outwardly-facing NTA residues of said liposomes; and
(iii) if necessary, removing excess protein by washing, filtering or other washing means and suspending the liposomes in an appropriate solution.
(iv) administering in vivo the engrafted liposomes containing the encapsulated/incorporated drug or agent to allow targeted delivery to a specific cell type or tissue for therapeutic purposes.

In a preferred embodiment, the molecules may be engrafted onto liposomes by the following method:-
(i) preparing a suspension of liposomes from a mixture of a phospholipid such as 1-palmitoyl-2-oleoyl-hosphatidylcholine (POPC) and a chelator lipid such as NTA-DTDA, in an aqueous solution such as PBS (phosphate buffered saline) containing a concentration of Nit²⁺ or Zn²⁺ approximately equal to that of the NTA-DTDA. The liposomes can be produced by sonicating the mixture for 5-10 mins at a temperature above the Tm. Alternatively, the liposomes can also be produced by dissolving the lipids in an ethanolic solution and then dispersing in aqueous buffer, or by extruding an acqeous suspension of the lipids through polycarbonate or similar filter of suitable pore size. Typically, the molar ratio of POPC:NTA-DTDA can be 10:1, and the total final lipid concentration can be ˜0.5 mM, but each can be different;
(ii) washing the liposomes by pelleting (by centrifuging at ˜95,000×g for 30 min at 4° C.) and removing the supernatant, or by filtration techniques, and then suspending the liposomes in an appropriate volume of the buffering solution to facilitate incubation with the tagged protein(s);
(iii) incubating the liposomes with a recombinant protein (e.g. human hex-ahistidine-tagged VEGF, vascular endothelial growth factor), or a combination of different recombinant proteins, each bearing a hexa-histidine or other suitable metal affinity tag to allow it to be anchored onto the liposomes; and
(iv) removing excess soluble or unincorporated protein by washing the liposomes as in step (ii) above, then suspending them in PBS or other buffer solution suitable for administration in vivo.

Different lipids or combinations of lipids can also be used in conjunction with the chelator lipid to give the liposomes specific properties. For example, the ganglioside GM1 or derivatives of polyethylene glycol can be included in the mixture (in step (i) above) to produce liposomes with “stealth” properties (9) to avoid them being taken up by macrophages or by the liver or spleen when used as vaccines in vivo. Also, step (i) can be carried out in the presence of a drug, DNA or other therapeutic agent for the purpose of encapsulating the material and permitting it, when administered in vivo, to be delivered to specific cells or tissues defined by the specificity of the engrafted molecule(s). For example, liposomes with engrafted VEGF (vascular endothelial growth factor) can be used to target angiogenic epithelium which is known to express the VEGF receptor and is required for tumor growth. Liposomes with engrafted VEGF, therefore, can be used to deliver a cytotoxic drug or agent that can block the growth of new blood vessels needed for the growth of tumors. The cytotoxic drug or agent is encapsulated within the liposome.

Examples of suitable molecules in accordance with this aspect of the present invention includes therapeutic molecules, pharmaceutical compounds and nucleic acid molecules such as RNA and DNA. A particularly useful molecule is VEGF or its homologue. VEGF and its homologues are also useful for targeting liposomes to cells carrying VEGF receptors. Accordingly, the molecules contemplated by this aspect of the present invention include molecules having binding partners on target tissue. Preferably, the molecules are engrafted onto liposomes that also contain encapsulated or incorporated a drug or therapeutic agent.

Examples of active material include, but are not limited to, a recombinant polypeptide, costimulatory molecule, therapeutic drug or nucleic acid molecule. In one example, VEGF is engrafted onto a liposome to target a cytotoxic drug to block the growth of new blood vessels needed for the growth of tumors.

Biological and/or synthetic membranes, liposomes or vesicles also can be engrafted with recombinant molecules for the purpose of developing vaccines and/or to produce a specific biological or therapeutic effect when administered in vivo.

Current methods of modifying the surfaces of cells to be used as vaccines for altering immunity to disease (e.g. the immune response to tumors—see below) generally require the transfection or genetic manipulation of the tumor cells, to induce them to express one or more specific protein(s) on their surface (10-12). For example, in both animal and human tumor models evidence suggests that the transfection of tumor cells with genes inducing them to express T cell costimulator molecules like B7-1 (CD80), B7-2 (CD86), CD40 and ICAM-1 on their surface, may be a useful approach to prepare the cells for use in vaccinations to enhance tumor immunity in the tumor bearing host (13-21). Unfortunately, in a clinical setting, such as in the treatment of cancer in humans, the transfection of tumor cells with such genes can be time consuming and inconvenient. Thus, the frequency of transfection is generally low, and successful transfection with multiple genes (to induce expression of multiple proteins on the tumor cell surface) can be difficult to achieve. Furthermore, transfection techniques, even when carried out by the use of seemingly harmless viral vectors, can be associated with risks to the patient owing to the difficulty in precisely controlling the expression of the gene or its integration into the genome.

The present invention further provides a more convenient and safe method of engrafting co-stimulatory and other molecules directly onto the surfaces of cells (such as tumor cells) and other membranous structures (either biological or synthetic), that can be used as vaccines to enhance or modify immunity to tumors and other diseases in humans.

Thus, the NTA-metal chelating linkage can be used to engraft molecules directly onto biological membranes (e.g. the membranes of cells or subcellular particles), once a chelator lipid (e.g. NTA-DTDA) has been incorporated into the membranes, thereby providing a convenient way of modifying the biological properties of these membranes.

A further aspect of the present invention thus provides for altering the immunogenicity of a target cell, or membranous component or structure. This may be readily accomplished by engrafting onto the cell or membranous structure foreign polypeptides, polysaccharides, glycoproteins, receptors, ligands and other molecules. Altering the immunogenicity of cells such as tumor cells, or components thereof, is a useful way of producing cell-based vaccines or agents that can enhance an immune response against tumor cells.

In this aspect of the present invention, there is therefore provided a method for altering the immunogenicity of a target cell or membranous component thereof, said method comprising engrafting a molecule onto the membrane of said target cell or component by:-
(i) preparing a suspension of chelator lipid or liposomes containing the chelator lipid;
(ii) incubating a suspension of cells or membranous structures with a suspension of the chelator lipid or liposomes containing the chelator lipid, to allow the chelator lipid to incorporate into the membrane of the cells or membranous structure;
(iii) if necessary, washing away excess or unincorporated lipid or liposomes;
(iv) incubating the cells or membranous structures with a solution of said molecule to be anchored; and
(v) if necessary, washing away excess or unbound soluble molecule, and suspending the cells or structures in a solution suitable for administration in vivo.

In a preferred embodiment, the invention allows for altering the immunogenicity of a target cell or membranous component thereof, using the following method:
(i) washing a suspension of the cells or membranous structures with PBS or other aqueous buffer solution to remove excess soluble and/or loosely bound proteins. This can be carried out by pelleting the structures by appropriate centrifugation (e.g. 5 min at 200-500×g for murine and human cells), and then resuspending them in PBS; depending on the structures, excess soluble or loosely bound proteins may be removed by filtering or other washing means;
(ii) preparing a suspension of chelator lipid (e.g. NTA-DTDA, at a concentration of ˜0.1 mM) in PBS containing an approximately equal concentration of either Zn²⁺ or Ni²⁺ by sonicating for 5-10 min an appropriate quantity of the lipid in the PBS solution. Other lipids or phospholipids (e.g. POPC) or other agents also can be included with the chelator lipid to promote the fusion and incorporation of the liposomes into the membrane structures;
(iii) incubating the cells or membranous structures with a suspension of the chelator lipid (e.g. 0.1 mM NTA-DTDA) in PBS for a suitable period of time and temperature (e.g. 30 min, at 37° C.) to allow some of the lipid in the suspension to fuse and/or become incorporated into the structures. Note: the incubation conditions employed can be altered to suit the nature of the chelator lipid used and the particular membrane structure into which the lipid is to be incorporated; also, incubations or wash steps in buffer containing additives such as polyethylene glycol can be used to promote lipid fusion and incorporation;
(iv) if necessary, removing unincorporated lipid from the mixture by washing the cells or membranous structures with PBS by pelleting and washing as in step (i) above;
(v) incubating the washed cells or membranous structures containing incorporated chelator lipid with a solution of a recombinant protein, or a solution of a mixture of different recombinant proteins, each containing a hexa-histidine or any other appropriate metal affinity tag; and
(vi) washing the cells or structures with PBS (as in step (i) above) to remove excess or unbound soluble recombinant protein.

A similar procedure can be used to alter the immunogenicity of any target cells, or subcellular membranous components thereof. The structures so treated will contain a modified surface due to incorporation of the chelator lipid and engraftment of protein, and can be used in vaccinations to alter immunological responses in vivo. These modified structures when administered in vivo also can be used to target a particular cell type or tissue within the body thereby inducing a response or altering the function of these cells or tissue. For example, the engrafting of tumor cells with molecules known to bind receptors on dendritic cells can be employed to direct the engrafted tumor cells to the dendritic cells to enhance tumor antigen presentation and hence immunological responses against the tumor.

In this form, the present invention contemplates a method of altering the biological and immunological properties of biological membranes, such as the membrane of cells and that of subcellular membranous components. In particular, the instant invention provides the basis of a convenient strategy for modifying the surfaces of cells (e.g. tumor cells), any cellular or subcellular membranous component, infectious agent or particle (e.g. bacteria), as well as any biological or synthetic membrane including synthetic vesicles or liposomes, into which the chelator lipid can be incorporated. In all these instances, the recombinant protein is engrafted by the formation of a metal chelating linkage between a peptide tag on the protein, and the NTA headgroup on the chelator lipid incorporated into the membranous structure. The biological membrane being modified by the anchoring of any recombinant protein, glycoprotein and any other molecular structure possessing an appropriate tag, and designed to enhance immunity to diseases when used either as a vaccine, or as an agent to target delivery of these biological membranes to specific cells and tissues when administered in vivo.

In a more particular embodiment, the present invention also describes the targeting of cells, biological and/or synthetic membranes or liposomes to a specific cell type or tissue within the body to achieve a therapeutic effect, said method comprising engrafting a molecule having a binding partner on the particular cell type or tissue to be targeted by:-
(i) preparing a suspension of chelator lipid or liposomes containing the chelator lipid;
(ii) incubating a suspension of cells or membranous structures with a suspension of the chelator lipid, to allow incorporation of the lipid;
(iii) if necessary, washing away excess or unincorporated lipid;
(iv) incubating the liposomes, cells, or membranous structures with a solution of molecules to be anchored; and
(v) if necessary, washing away excess or unbound soluble molecule; then suspending the liposomes, cells, or structures in a solution suitable for administration in vivo.

Accordingly, the present invention enables the incorporation of chelator lipids like NTA-DTDA into tumor cell membranes, followed by the engraftment of recombinant co-stimulatory and/or other molecules (or combinations of molecules) with an appropriate tag, may be a convenient approach in the development of cell-based vaccines to enhance tumor immunity. Analogous to its demonstrated ability to alter tumor immunity therefore, the technique also can be expected to provide a convenient approach to engraft specific co-stimulatory and/or other cell surface molecules (or combinations of such molecules) onto other cell types including T cells, B-cells and dendritic cells, to see what role such molecules might play in regulating immune function. In addition to its potential use in cancer immunotherapy, therefore, the technique described herein will have application to areas that could significantly enhance our understanding of immune function.

Yet another aspect of the present invention provides method of modifying biological and/or synthetic membranes and liposomes to achieve a specific therapeutic effect, such as the induction or modulation of an immune response or other physiological or biological response, when administered in vivo, said method comprising:-
(i) preparing a suspension of chelator lipid or liposomes composed of a mixture of lipids and the chelator lipid; or
(ii) incorporating the chelator lipid onto the cells or membranes, by incubating a suspension of the cells or membranes with a suspension of liposomes containing the chelator lipid, and if necessary, washing away excess or unincorporated lipid or liposomes;
(iii) incubating the liposomes, cells or membranous structures with a solution of a recombinant protein(s) or target molecule(s) possessing an appropriate metal affinity tag; and
(iv) washing away excess or unbound soluble protein, and suspending the liposomes, cells, or membranous structures in a solution suitable for administration in vivo.

As stated above, the present invention provides methods for altering the immunogenicity of cells. Accordingly, the present invention provides a method of treatment, said method comprising administering to a subject an effective amount of a liposome preparation or membranous material comprising an active material and optionally an anchored or engrafted molecule having a binding partner or target tissue.

More particularly, the present invention provides a method of treatment, said method comprising administering to a subject an effective amount of a liposome preparation or membranous material comprising an encapsulated or incorporated drug or active material, and an engrafted targeting molecule having a binding partner on the particular cell type or tissue to be targeted in vivo.

Another aspect of the present invention provides a vaccine composition comprising cells or membranous material having engrafted thereto molecules capable of modifying an immunological response to a subject to which the vaccine is administered, said vaccine further comprising one or more pharmaceutical carriers and/or diluents. Preferably, the molecules engrafted to the cells or membranous material are co-stimulatory molecules. Furthermore, the vaccine is preferably produced by the steps comprising:-
(i) incubating the cells or membranous material with a chelator lipid such as NTA-DTDA to allow the lipid to incorporate in the cells or membranes;
(ii) washing off any unincorporated lipid by centrifugation or filtration and resuspension of the structures in the appropriate solution or buffer;
(iii) incubating the membranous structures with incorporated chelator lipid with said molecules to be engrafted; and
(iv) washing off unincorporated molecular material.

In a related embodiment, the present invention provides a vaccine composition comprising cells, liposomes, vesicles or membranous material having engrafted thereto molecules capable of modifying an immunological response to a subject to which the vaccine is administered, said vaccine further comprising one or more pharmaceutical carriers and/or diluents.

The present invention further provides a use of membrane system comprising an agent engrafted, encapsulated and/or anchored thereto in the manufacture of a medicament for modifying an immune response in an animal.

Preferred animals in accordance with the present invention are humans, primates, livestock animals, laboratory test animals and captured wild animals.

Terms such as “anchoring” and “engrafting” may be used interchangedly throughout the subject specification. The term “engrafting” also encompasses the term “grafting”. The terms “membrane” and “lipid membrane” include reference to biological and synthetic membranes as well as lipid layers. A “chelator lipid” may be any suitable chelating lipid such as but not limited to NTA-DTDA.

Still another aspect of the present invention contemplates a method of targeting cells biological and/or synthetic membranes and liposomes to a particular cell type or tissue within the body, said method comprising engrafting onto the membrane structure a molecule having a binding partner on the particular cell or tissue to be targeted by:-
(i) preparing a suspension of chelator lipid or liposomes containing the chelator lipid; or
(ii) incubating a suspension of the cells or membranous structures with a suspension of the chelator lipid to allow the lipid to become incorporated;
(iii) if necessary, washing away excess or unincorporated lipid;
(iv) incubating the liposomes or membranous structures with a solution of molecules to be anchored; and
(v) if necessary, washing away excess or unbound soluble molecule, and suspending the liposomes or structures in a solution suitable for administration in vivo.

The materials and methods below relate to some of the Examples which follow.

Reagents

Analytical grade reagents were used in all experiments. Paraformaldehyde was obtained from BDH Chemicals. ZnSO₄ was used for all additions of Zn²⁺ to buffers and growth media. RPMI 1640 and EMEM (Eagles minimal essential medium) both were obtained from Gibco (Life Technologies, Melbourne, Australia). Fetal calf serum (FCS) was obtained from Trace Scientific (Noble Park, Vic. Australia). Sulfo-NHS-LC-Biotin was obtained from Pierce (Rockford, Ill.). Na⁵¹CrO₄, [³H]-thymidine, and fluorescein isothiocyanate (FITC)-conjugated streptavidin (streptavidin-FITC) were obtained from Amersham (UK). Dioleoyl-phosphatidylethanolaraine (DOPE), α-palmitoyl-β-oleoylphosphatidylcholine (POPC), dimyristoyl-phosphatidylcholine (DMPC), Isopaque, ficoll, propyl gallate, and the polyethylene glycol (PEG) preparations PEG₄₀₀, PEG₆₀₀, PEG₉₀₀, and PEG₁₅₀₀, were all obtained from Sigma-Aldrich Pty Ltd (Castle Hill, NSW, Australia). MicroScint scintillation fluid and other items such as filters and seals for 96-well plates for use with the Topcount NXT microplate scintillation counter were obtained from Canberra Packard (Canberra, ACT, Australia).

Mice and Cell Lines

Female or male DBA/2J mice (H-2^{d}), were used for isolation of lymphoid tissue (spleen) for T cell proliferation, measurement of T cell cytotoxicity, and for vaccination and monitoring of tumor growth in vivo. C57BL/6J mice (H-2b) were used in experiments assessing allogeneic stimulation of T cell proliferation. The mice were used at 6-8 weeks of age and were obtained from the Animal Breeding Establishment, John Curtin School of Medical Research (JCSMR), Australian National University (ANU), Canberra. The murine cell lines, P815 [murine DBA/2 (H-2^{d}) mastocytoma] and EL4 [murine C57BL/6 (H-2^{b})] T cell lymphoma, were obtained, respectively, from Drs P. Waring (Division of Immunology and Cell Biology, JCSMR) and H. O'Neill (Division of Biochemistry and Molecular Biology), ANU. Both cell lines were cultured in complete medium consisting of EMEM containing 10% v/v FCS.

Synthesis of NTA DTDA

The chelator-lipid nitrilotriacetic acid didtetradecylamine (NTA-DTDA), consisting of a nitrilotriacetic acid (NTA) head-group covalently linked to ditetradecylamine (DTDA) was synthesized by Dr C. Easton (Research School of Chemistry, ANU) following a procedure similar to that previously described (22). Briefly, the DTDA was synthesized from bromotetradecane and ammonia. DTDA was then N-succinylated with succinic anhydride to produce N-succinyl-DTDA (DTDA-suc), which was reacted with N-hydroxysuccinimide (NHS) to produce N-[(hydroxysuccinimidyl)succinyl]-DTDA (DTDA-suc-NHS). The succinimidyl group of DTDA-suc-NHS was replaced with a N^{α}-tert-butyloxycarbonyl-lysine (N-Boc-lys) group, and the butyloxycarbonyl (Boc) group was removed to produce N^{ε}[(DTDA) succinyl]-L-lysine (DTDA-suc-Lys). DTDA-sucLys was finally reacted with bromo-acetic acid to produce N^{α},N^{α}bis[carboxymethyl]-N^{ε}[(DTDA)suc]-L lysine, which will be referred to as NTA-DTDA. The purity of each product was measured by thin layer chromatography, and the identity of the final product was confirmed by nuclear magnetic resonance spectroscopy, Fourier transformed infrared spectroscopy and mass spectroscopy. The purity of the final product was estimated to be in excess of 99%.

Preparation of NTA-DTDA Liposome Suspensions

For NTA-DTDA incorporation into cells, dessicated NTA-DTDA was suspended to a concentration of 0.5 mM in PBS containing 0.5 mM Zn²⁺, by sonication using a TOSCO 100W ultrasonic disintegrator at maximum amplitude for 2 min. The same procedure was used to produce suspensions of DMPC, and mixtures of NTA-DTDA and DMPC, POPC, or DOPE. Stock suspensions of lipids were stored at −20° C., and were always re-sonicated and diluted to the indicated concentration prior to use in experiments.

Monoclonal Antibodies

The monoclonal antibodies (mAb) and their sources were as follows: murine anti-CD40 (clone 3/23, Rat IgG₂ₐ) and murine anti-CD3 (clone 145-2CI1, Armenian Hamster IgG) mAbs were both obtained from Pharmingen; and murine anti-B7.1 (clone 16-10A1, Armenian Hamster IgG) mAb was from The Walter and Eliza Hall Institute of Medical Research, Melbourne, Australia. Where indicated, mAbs were biotinylated by reacting with sulfo-LC-biotin (Pierce) as previously described (23).

Recombinant Proteins

Recombinant forms of the extracellular regions of the murine T cell co-stimulatory molecules B7.1 (CD80) and CD40, and the extracellular region of the human erythropoietin receptor (EPOR), each with a hexahistidine (6His) tag and denoted B7.1-6H, CD40-6H, and EPOR-6H, respectively, were produced using the baculovirus expression system. Briefly, genes encoding the extracellular domains of murine B7.1, CD40 and EPOR were amplified by polymerase chain reaction (PCR), and the sequences for 6His tags were incorporated into the end of each gene (corresponding to the carboxyl terminal of the protein) by PCR using primers containing the sequence of the tag. The constructs were then separately ligated into the pVL1393 plasmid baculovirus transfer vector and used to transform E. coli. Appropriate transformants were selected, and recombinant pVL1393 plasmids from these transformants were co-transfected with the baculovirus AcMNPV into SF9 insect cells. Cells infected with virus which had the pVL1393 plasmid incorporated into the viral genome were selected by plaque assays, further amplified and these viral stocks were used to infect High-5 insect cells grown in Express-5 medium. Recombinant proteins were purified from the supernatants of recombinant virus infected High-5 cells by Ni²⁺-NTA affinity chromatography (using Ni²⁺-NTA Superflow, from QIAGEN Pty Ltd, Cifton Hill, Victoria, Australia) followed by size exclusion gel filtration on FPLC (Pharmacia Biotech, Upsalla, Sweden) using a Superdex-75 HR 10/30 column; the final purity of each protein was >95% as judged by SDS-PAGE analysis. For some experiments recombinant proteins were biotinylated by reacting with sulfo-LC-biotin (Pierce) as previously described (23). The proteins were routinely stored at −20° C. in PBS at a concentration of 0.2-0.6 mg/ml, and then thawed at 37° C. and vortexed gently prior to use in each experiment.

Incorporating and Optimizing the Incorporation of NTA-DTDA

Cultured P815 tumor cells were washed twice in PBS to remove proteins from the culture media and suspended to 1×10⁷ cells/ml in PBS. The cells were then aliquoted into 96-well V-bottom Seroeluster plates (Costar, Corning, N.Y.) at 1.8×1⁵ cells/well and incubated with 125 μM NTA-DTDA (alone or as a mixture with other lipids as indicated) or 125 μM DMPC (control) in PBS containing 125 μM Zn²⁺, for 40 min at 37° C. Following the incubation, unincorporated lipid was removed by washing three times with PBS containing 0.1% v/v BSA (PBS-0.1% v/v BSA). The relative level of NTA-DTDA incorporated was routinely assessed by FACS analysis (see below) after incubating the cells with biotinylated 6His peptide (B-6His) (0.2 μg/ml) for 30 min at 4° C., washing twice with PBS-0.1% v/v BSA, and then staining with streptavidin-FITC. The cells were incubated with streptavidin-FITC (33 μg/ml) in PBS containing 1% v/v BSA (PBS-1% v/v-BSA) for 30 min at 4° C., washed three times with PBS-1% v/v BSA, fixed with 2% v/v paraformaldehyde in PBS, and then analyzed for FITC-fluorescence by FACS.

To promote fusion of NTA-DTDA liposomes and hence incorporation of the NTA-DTDA into the membrane of cells, a number of agents previously reported to potentiate the fusion of cells and vesicles with lipid layers were tested. P815 cells aliquoted into 96-well V-bottom serocluster plates as described above were incubated with 125 μM NTA-DTDA, DMPC, POPC, or DOPE, or with 125 μM NTA-DTDA plus DMPC, POPC or DOPE (at the indicated molar ratio), in PBS containing 125 μM Zn²⁺, for 40 min at 37° C. For some experiments the cells were treated with PEG following the incubation: the cells were pelleted, suspended in 15% PEG₄₀₀, mixed and diluted 10× with serum-free EMEM, and then washed once with serum-containing EMEM and twice with PBS-0.1% v/v BSA, before engrafting the cells with biotinylated recombinant protein (see below) and then staining with streptavidin-FITC as above for FACS analysis.

Engrafting Recombinant Proteins onto Cells

Cells with incorporated NTA-DTDA were incubated with purified B7.1-6H and CD40-6H (or biotinylated forms of these as indicated), either alone (each at 50 μg/ml) or in combination (100 μg/ml total protein, with a B7.1-6H:CD40-6H molar ratio of 4:1), in 96-well V-bottom Serocluster plates for 1 hr at 4° C. Unbound protein was then removed by washing twice with PBS-0.1% v/v BSA, before using the cells bearing the engrafted protein(s) either for immunizations or in assays of T cell proliferation. For experiments to determine the level of bound protein by FACS analysis the cells were stained with streptavidin-FITC (for cells bearing engrafted biotinylated protein), or were first incubated with the appropriate biotinylated mAb (B-mAb) (4° C. for 30 min), washed twice with PBS0.1% w/v BSA and then stained with streptavidin-FTTC.

Time Courses

Cells with incorporated NTA-DTDA and DMPC, with or without engrafted CD40-6H, were suspended in EMEM containing 10% v/v FCS and 50 μM added Zn²⁺, and incubated in 12-well flat-bottom tissue culture plates (Linbro, ICN Biomedicals Inc, Aurora, Ohio) for approx. 2 min (time 0), or 4 or 24 hrs at 37° C. After the indicated incubation time, cells were collected from the 12-well flat bottom plates, transferred to 96-well V-bottom Serocluster plates and washed twice in PBS-0.1% v/v BSA, before either staining with streptavidin-FITC (for cells with NTA-DTDA and engrafted B-CD40-6H), or first incubating with B-CD40-6H and then washing with PBS-0.1% v/v BSA and staining with streptavidin-FITC (for cells with only NTA-DTDA).

Flow Cytometry

Fluorescence-activated cell sorter (FACS) analysis was used to quantify the relative levels of NTA-DTDA incorporated into the membrane of cells following binding of B-6His, and the levels of biotinylated recombinant proteins anchored to the cell surface via the incorporated NTA-DTDA. Flow cytometric analyses were performed using a FACSort flow cytometer (Becton Dickinson, San lose, Calif.) equipped with a 15 mW argon-ion laser. Cells were analysed on the basis of forward light scatter (FSC), side light scatter (SSC) and FITC-fluorescence; with the relative shift in fluorescence intensity above background providing a semi-quantitative measure of the level of NTA-DTDA incorporation and the level of peptide or recombinant protein on the surface of cells. Typically, fluorescence information for 10,000 cells was collected for each condition using a log amplifier and the data processed using CELLQuest (Becton Dickinson) software. Data were analyzed by gating live cells, as judged by FSC versus SSC dot plots, and plotting the fluorescence profile as a histogram. The fold increase in fluorescence intensity above background was determined by measuring the shift in fluorescence intensity, using the control sample as background, from peak to peak. The results of independent experiments were then represented as the mean±the standard error of the mean (SEM).

Confocal Microscopy

The distribution of the NTA-DTDA on the surface of P815 cells was studied by laser scanning confocal microscopy using cells bearing incorporated NTA-DTDA engrafted with biotinylated CD40-6H, and stained with streptavidin-FITC. Briefly, the cells were suspended in embedding medium (2% propyl gallate in 87% v/v glycerol) and deposited into 0.05 mm deep chambers on microscope slides formed using perforated Scotch 465 adhesive transfer tape, and the chambers were then sealed with glass cover slips. The cells were examined for fluorescence at 520 nm with a MRC-500 Laser Scanning Confocal Imaging System (BioRad), consisting of a Nikon confocal fluorescence microscope (×60 Nikon objective), with a BioRad UV-laser scanner and an Ion Laser Technology laser head (model 5425, BioRad) with an argon ion laser. The image was acquired by Kalman averaging of 10 successive laser scans, and stored and analyzed using Image Processor PC (BioRad) and processed using NIH Image 1.61 software.

T Cell Proliferation Assays

Murine T cells for use in T cell proliferation assays were isolated and purified from the spleens of either allogeneic or syngeneic mice as described (24). Briefly, the spleens were dissociated into single cell suspensions, and dead cells and red blood cells were removed by density gradient centrifugation using an Isopaque-Ficoll gradient. After centrifugation (20 min at 400×g) the viable cells, mainly lymphocytes, were collected from the layer at the top of the gradient and suspended in RPMI 1640 containing 10% v/v FCS, 5×10⁻⁵ M 2-β-mercaptoethanol, 100 i.u./ml penicillin, 100 μg/ml neomycin, IL-2, and 10 mM HEPES. T cells were purified using an equilibrated nylon wool column (25). The purified T cells were then suspended in growth medium at a concentration of 2×10⁴ cells/50 μl/well in a 96-well flat bottom plate (Cell Wells, Corning, N.Y.) for culture at 37° C. in an atmosphere of 5% CO₂.

T cell proliferation assays were carried out as described (25). Syngeneic lymphocytes or responder cells were then co-cultured with γ-irradiated (5000 rad) stimulator cells at a concentration of 2×104 cells/50 μl/well. Stimulator cells included native P815 tumor cells, P815 cells with incorporated NTA-DTDA on their surface, and P815 cells with engrafted recombinant protein(s), as indicated. After 4 days co-culture at 37° C., the cells were pulsed with 1 μCi of [³H]-thymidine per well for 6 hrs. The cells were then harvested using a Filtermate 196 cell harvester (Packard) and [³H]-thymidine incorporation assessed using MicroScint scintillant and a Topcount NXT microplate scintillation counter (Packard) using Topcount software.

Cytotoxicity Assays

Assays for in vivo tumor-specific CTL were performed by a procedure similar to that described by Chen et al. (26). Syngeneic DBA/2 mice were immunized subcutaneously with either PBS (control) or 1×10⁵ γ-irradiated (5000 rad) P815 cells engrafted with recombinant protein(s). Spleens were removed from mice 14 days after immunization, and T lymphocytes (effector T cells) were isolated by density gradient centrifugation using Isopaque-Ficoll and nylon wool fractionation, as described above. Effector T cells were then suspended in incubation medium and aliquoted into 24-well flat-bottom plates at a concentration of 1×10⁵ cells/well and co-cultured with 1×10⁵ γ-irradiated (5000 rad) native P815 cells. After 5 days of co-culture, the cytolytic activity of the effector cells was assessed in a standard ⁵¹Cr-release assay, as described (26). Briefly, 2×10⁶ native P815 cells were labelled with 250 μCi ⁵¹Cr (Na⁵¹CrO₄) for 90 min. Labelled target cells were washed three times and resuspended in culture medium. Effector and target cells were coincubated with effector cells at different effector to target ratios, as indicated, for 6 hrs at 37° C. Supernatants were harvested and ⁵¹Cr release assessed with a Topcount NXT microplate scintillation counter (Packard) using Topcount software (Packard). Percent specific lysis was calculated as follows:

% Specific lysis=100×(experimental cpm−spontaneous cpm) maximal cpm−spontaneous cpm)

Immunization of Animals and Tumor Challenge in vivo

Mice were immunized using a protocol similar to that described (26). Briefly, either PBS (control) or 1×10⁵ γ-irradiated P815 cells with the engrafted recombinant protein(s) as indicated, were suspended izi a 0.2 ml volume of PBS and injected into the shaved right back of syngeneic DBA/2 mice using a 25-gauge needle and 1 ml syringe. After 14 days the mice were either used in cytotoxicity assays using T cells isolated from the spleens of the mice, or were challenged with 1×10⁵ native P815 cells by subcutaneous injection in the shaved left back. For monitoring tumor growth, the mice were scored for tumor size once a week by measuring two perpendicular diameters in millimeters using a caliper (26). Survival data represent animals that were still alive when scored; animals that were near death were euthanized after scoring and were deemed to have died of the tumor. Data for a total of 10 or 12 mice in the group for each experimental condition is presented.

### Example 1

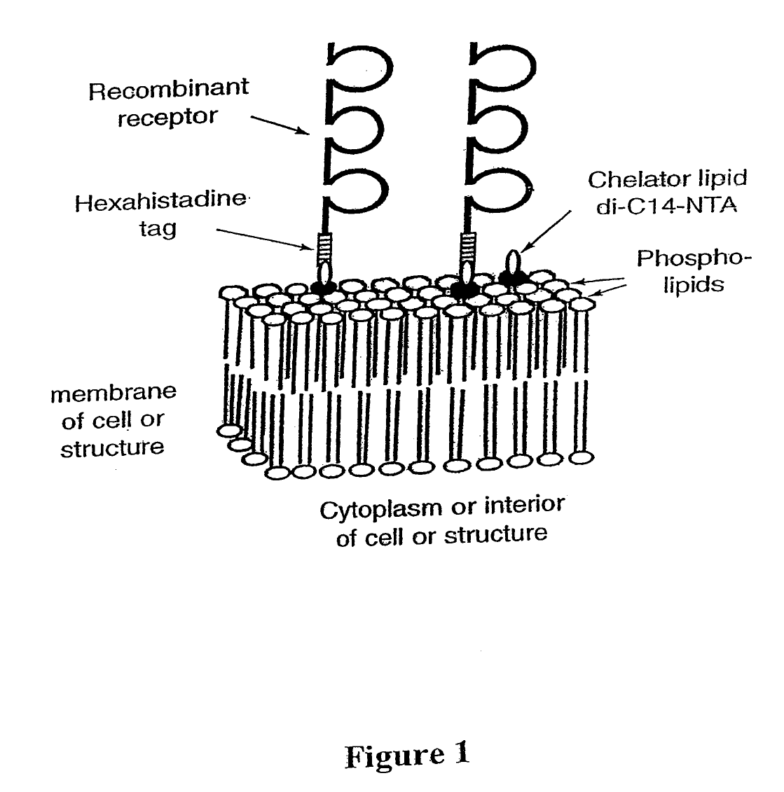

Using the instant invention to modify the surface of cells and other biological and/or synthetic membranes and liposomes by engraftment of hexahistidine-tagged molecules (see FIG. 1), for the development of vaccines and for drug targeting in vivo.

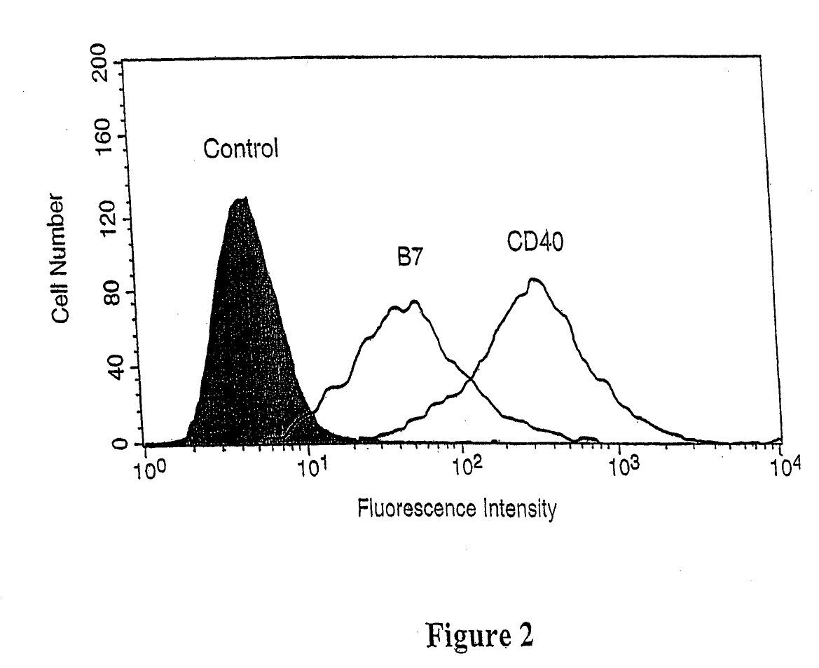

The histograms in FIG. 2 show fluorescence-activated cell sorting (FACS) profiles of murine mastocytoma P815 cells carrying engrafted recombinant hexa-histidine-tagged murine B7.1 and CD40. P815 cells were pre-incubated for 30 min at 37° C. with a suspension (0.1 mM) of control lipid di-myristoyl-phosphatidylcholine (DMPC; also referred to as di-C14-PC; control), or the chelator lipid NTA-DTDA, before being washed in PBS and incubated with a mixture of hexa-histidine-tagged B7.1 and CD40 (each at ˜20 μg/ml). The cells were then washed again in PBS and stained by an incubation (30 min at 4° C.) with either biotinylated 16-10A1 or biotinylated B-3/23 monoclonal antibody (i.e. biotinylated anti-B7.1 or anti-CD40), as indicated, followed by an incubation with FITC-conjugated streptavidin. Cells incubated with DMPC and recombinant proteins show a low level of fluorescence after staining with either monoclonal antibody (Control). The fluorescence of P815 cells pre-incubated with NTA-DTDA is 10-100-fold higher than that of cells pre-incubated with DMPC (Control). Each result is a representative of two experiments performed in duplicate. The results show that chelator lipids (in this instance NTA-DTDA) can be incorporated into the membrane of these cells, and that the incorporated lipid can be used to anchor or engraft hexa-histidine tagged B7.1 and CD40 directly onto the P815 cell surface via the NTA-DTDA. In other studies we showed that recombinant murine B7.1 and CD40 bearing a hexa-histidine tag can be engrafted onto the surface of all the different cell lines tested; these included murine P815 and EL4 tumor cells, human leukemic Jurkat cells and yeast cells.

### Example 2

The Example relates to modifying the surface of tumor cells to enhance tumor immunity.

Recent work indicates that the transmembrane and cytoplasmic regions of B7-1 and B7-2 are not required for T cell co-stimulation (20), and that T cell co-stimulation also occurs when the B7-1 is expressed on tumor cell surfaces in a GPI-anchored form (21). Also, the extracellular regions of any cell surface receptor molecules (e.g. the murine T cell co-stimulator molecules B7.1 and CD40) can be produced to contain a hexa-histidine or other appropriate peptide tag on the carboxyl terminal. In this form the present invention provides a method of anchoring these co-stimulator molecules directly onto the cell surface in the correct orientation, thereby mimicking the co-stimulatory function of these molecules on the surface of antigen presenting cells. The instant invention, therefore, has implications for tumor vaccine development, by providing a more convenient and safe alternative to transfection for putting co-stimulator and/or other relevant molecules onto tumor cells for use in immunizations to enhance immunity to tumors.

The viability of using engrafted molecules can be tested by assaying for functional responses dependent on the engrafted molecules. Thus, the ability of murine P815 mastocytoma (DBA/2, H-2d) cells carrying engrafted hexa-histidine tagged B7-1 and/or CD40 to stimulate a T cell proliferative response in an allogenic system was examined using splenocytes isolated from C57B1/6 (H-2b) mice co-cultured with an appropriate number of γ-irradiated P815 cells (as control), or P815 cells engrafted with hexa-histidine-tagged B7-1 and/or CD40. Preliminary experiments in which the incorporation of 3H-thymidine was used to measure T cell proliferation, show that the P815 cells bearing engrafted hexa-histidine tagged B7-1 and/or CD40 are able to stimulate an increased level of T cell proliferation in this mixed cell reaction. These results are consistent with the invention being useful to modify cells for use in vaccinations to enhance anti-tumor responses.

### Example 3

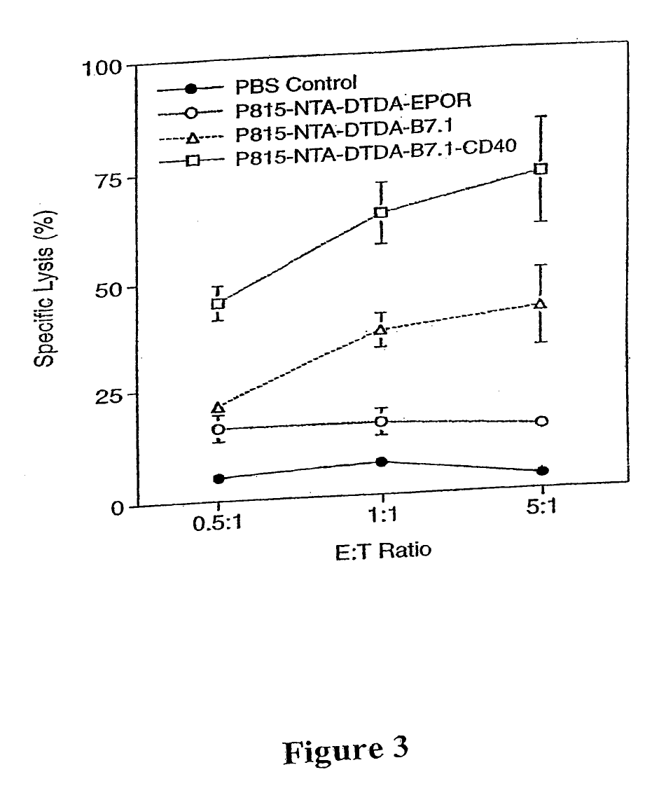

To test the ability of P815 cells bearing engrafted co-stimulatory molecules to induce anti-tumor responses in vivo, mice were immunized with P815 cells bearing the engrafted molecules to see if this could stimulate CTL activity and/or affect tumor growth in syngeneic animals. Separate groups of DBA/2 mice were immunized with either PBS, or with γ-irradiated P815 cells bearing engrafted EPOR-6H, B7.1-6H, or B7.1-6H plus CD40-6H. Two weeks after immunization, spleens were removed from the mice, and splenic T cells were isolated and assessed for their ability to kill native P815 cells in a standard ⁵¹Cr release assay. The data in FIG. 3 show that at all the effector target cell ratios indicated (0.5:1, 1:1, 5:1), only a low level (2-5%) of lysis was induced by T cells from mice immunized with PBS (as control). The lytic activity of T cells from mice immunized with P815-EPOR (as control protein) was also low ranging from 7-16%. Interestingly, at all effector:target cell ratios tested, the level of tumor cell-specific lysis was higher for conditions where the effector T cells were derived from mice immunized with P815 cells bearing one or more engrafted co-stimulatory molecule(s) (see FIG. 3). The highest cytolytic activity was observed at the effector:target cell ratio of 5:1, for which the specific lysis induced by T cells obtained from mice immunized with P815 cells bearing engrafted B7.1, and P815 cells with engrafted B7.1 and CD40, was 3- and 5-fold higher, respectively, than that for T cells obtained from mice immunized with P815 cells engrafted with control protein (see FIG. 3). Parallel experiments using native EL4 cells instead of P815 cells showed only background levels of lysis, indicating that the cytolytic response was specific for P815 cells as targets. The results indicate that CTL responses against P815 cells can be generated in mice immunized with P815 cells bearing engrafted B7/CD40.

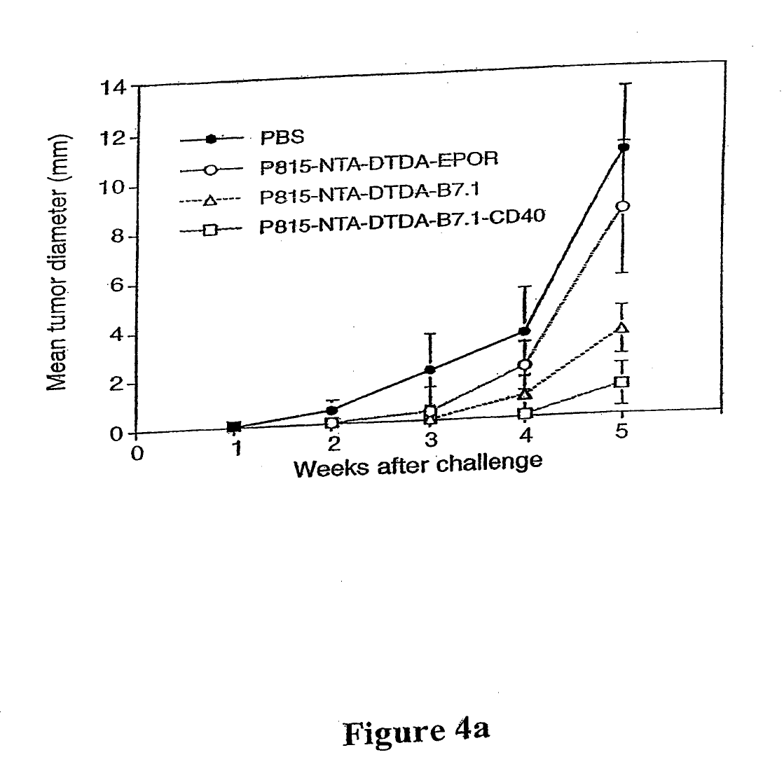

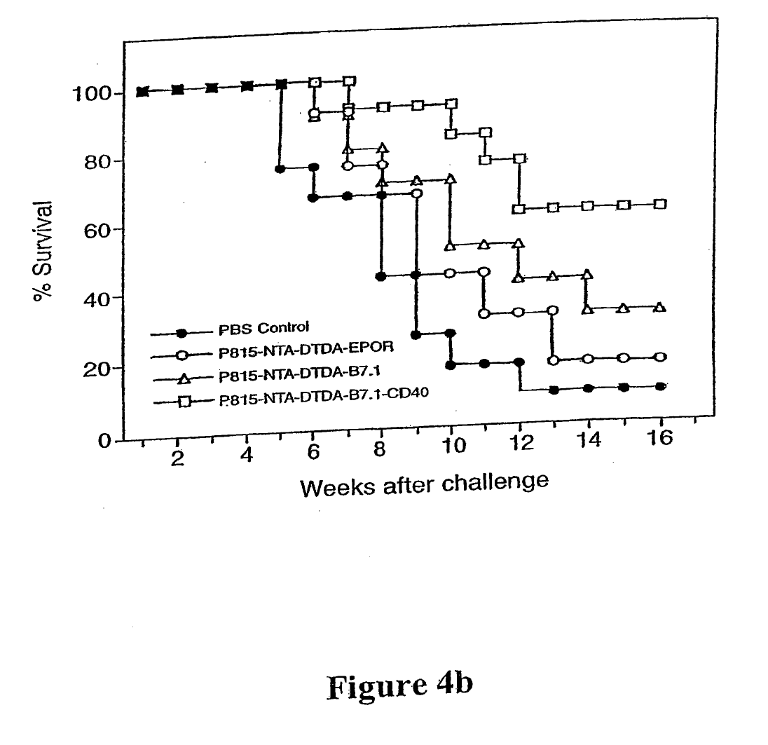

To determine whether the immunization of mice with P815 cells bearing engrafted co-stimulatory molecules could induce tumor immunity, groups of mice immunized with γ-irradiated cells bearing the engrafted proteins, also were monitored for tumor growth and survival after a challenge with native P815 cells. These studies indicated a slower rate of tumor growth in mice immunized with P815 cells bearing engrafted co-stimulatory molecule(s), compared to mice immunized with cells bearing control protein. Thus, at 5 weeks after tumor challenge the mean tumor diameter was 3.36±1.0 mm and 1.1±0.9 mm, for mice immunized with P815 cells bearing engrafted B7.1-6H and B7.1-6H plus CD40-6H, respectively; and 10.7±2.5 mm and 8.3±2.7 mm for mice immunized with PBS and P815 cells engrafted with EPOR-6H, respectively. Tumor growth data as reflected by the mean tumor diameter for only the first 5 weeks after challenge is presented since from this time some animals died from the tumor. At 14 weeks after tumor challenge survival was ˜17% for control mice, ˜30% for mice immunized with P815 cells engrafted with B7.1, and ˜60% for mice immunized with P815 cells engrafted with both B7.1 and CD40 (see FIGS. 4(a) and (b)). Consistent with the observed increase in CTL activity, the results indicate that the immunization of syngeneic animals with P815 cells bearing engrafted co-stimulatory molecules can inhibit tumor growth and prolong survival of the animals after a challenge with the native P815 tumor.

### Example 4

Vascular endothelial growth factor (VEGF) is a homodimeric glyocoprotein hormone of ˜40 kDa that is among the most potent of angiogenic mitogens, and a major regulator of angiogenesis (27). Considerable evidence suggests that VEGF is secreted by tumor cells and other cells exposed to hypoxia, and that VEGF is a major angiogenic factor in solid tumor development (28). In addition to its potent angiogenic activity, VEGF increases vascular permeability and favours the migration of endothelial cells through the extravascular matrix, processes that are essential for tumor angiogenesis, tumor spread, and metastasis (29). Human VEGF is known to stimulate endothelial cell growth and differentiation by binding to high affinity VEGF receptors such as the kinase domain receptor (human KDR, or murine flk-1), and the Fms-like tyrosine kinase-1 (Flt-1). These receptors are expressed exclusively on proliferating vascular endothelial cells, and their expression is known to be increased by a number of factors often produced by tumors (27-30). That VEGF and its receptors are important for tumor growth has been demonstrated by the fact that the neutralization of VEGF by the use of antibodies (31) or recombinant soluble receptor domains (32) exhibit therapeutic potential as agents that can suppress tumor growth and metastasis in vivo.

The substantially exclusive expression of VEGF receptors on proliferating endothelial cells, suggests the VEGF receptor can be used as a targeting molecule in therapeutic strategies that target neovascularization. The development of sterically stabilized or “stealth” liposomes (SLs) which can evade elimination by the phagocytic cells of the immune system (e.g. the reticulo-endothelial system in the liver and spleen), recently has provided a major advance for liposomal drug delivery in cancer chemotherapy (33-35). In contrast to conventional liposomes which are cleared rapidly from the blood (often within minutes) after their administration in vivo, SLs remain in the blood circulation for several days. A number of studies have demonstrated therapeutic benefit of SLs containing an encapsulated cytotoxic drug like doxorubicin, in the treatment of AIDS-related Kaposi sarcomas and other lesions characterized by leaky vasculature (35). Recent studies also describe the targeting of SLs to specific tumors, with the targeting being achieved primarily by the use of “immuno liposomes”, or liposomes with specific antibody (or F(ab′)₂ fragments) covalently attached to the liposome surface (36). The immobilization of targeting proteins such as antibodies onto SLs encapsulated with doxorubicin apparently does not alter their stealth-like characteristics, but can endow the liposomes with specific targeting properties (37-40).

Until the advent of the present invention, the coupling of target molecules to liposomes has been difficult and there is potential to induce unwanted anti-idiotypic responses to the antibody used. In accordance with an aspect of the present invention, two chelator lipids, nitrilotriacetic acid di-tetra-deeylamine (NTA-DTDA) and nitrilotriacetic acid polyethyleneglycol (2000) phosphatidylethanolamine (NTA-PEG2000-PE), are used with a recombinant form of VEGF, to develop SLs containing encapsulated doxorubicin that will target and specifically destroy proliferating vascular endothelial cells in vivo, thereby blocking neovascularization and tumor growth.

VEGF is a particularly attractive targeting molecule as VEGF receptors are expressed substantially exclusively on angiogenic endothelium. In accordance with the present invention, it is proposed to produce a “stealth” liposome containing encapsulated doxorubicin and surface VEGF anchored through NTA-lipids like NTA-DTDA. It is proposed in accordance with the present invention that when administered in vivo, this agent will target and destroy proliferating endothelial cells, and will inhibit tumorigenesis and tumor metastasis.

Liposomes composed largely of conventional lipids like egg-yolk phosphatidylcholine (PC) and cholesterol (Chol), and a small proportion (˜10%) of a sterically “stabilizing” lipid such as ganglioside GM1 (32), or a phosphatidyl-ethanolamine conjugated to polyethyleneglycol (2000) (PEG₂₀₀₀-PE) (33, 34), are reported to exhibit increased stability and prolonged circulation times in blood, largely escaping elimination by the reticulo-endothelial system. These properties of SLs have been attributed to the presence of lipids possessing uncharged headgroups which increase interaction with water, but inhibit interaction with either charged or hydrophobic structures likely to be encountered on proteins and cells in plasma (32, 33). Evidence suggests that SLs (or immunoliposomes) with antibody molecules attached to the distal end of the PEG chains on the SL surface, interact more effectively with their target than liposomes with the antibody attached directly onto the SL surface. This has been explained by the PEG chains sterically interfering with the ability of the antibody to interact with antigen under these conditions (37). SLs made using ganglioside GM1, or a PEG-lipid with a shorter PEG chain length (eg. PEG750-PE, rather than PEG2000 PE), therefore, are likely to be more suited for the binding of 6His-VEGF directly to the NTA-DTDA on the liposome surface, and for optimal binding of the engrafted VEGF to VEGF receptors on target cells. Ganglioside GM1 and PEG750-PE are both commercially available (from Avanti Polar Lipids), and each will be tested with liposomes made from PC, Chol and NTA-DTDA. To further reduce possible steric effects the inventors produce a novel lipid, namely NTA-PEG2000-PE, which consists of one or more NTA groups attached to the distal end of the PEG chain on the PE. This compound, is used in combination with PEG2000-PE to produce SLs which allow convenient engraftment of targeting 6His-proteins (such as 6His-VEGF), while eliminating the possibility of steric hindrance. This is approach greatly facilitates the use of SLs in therapeutic applications requiring their targeting to specific cells and/or tissues.

Recombinant 6His-VEGF is produced. SLs are produced from a mixture of lipids including PC, Chol, NTA-DTDA, and “stealth” lipids such as ganglioside GM1, PEG2000-PE and NTA-PEG2000-PE. The SLs are engrafted with 6His-VEGF (VEGF-SLs) and then assessed for their ability to target endothelial cells. Conditions for specific binding of the liposomes to proliferating endothelial cells in culture are optimized, relative to their binding to cells that lack the VEGF receptor. The specific cytoxicity of VEGF-SLs encapsulated with doxorubicin will be assessed using human vascular endothelial cells in vitro. VEGF-SLs intrinsically labelled with fluorescent dyes and/or radioactive tracers are administered intravenously into mice to determine their distribution in various tissues with time. The proportion of each stabilizing lipid used for producing SLs is altered to optimize the “stealth” properties of the liposomes, as judged by a reduction in the proportion of the liposomes taken up by the liver, spleen and other major organs, relative to vascularizing tumors. As the VEGF receptor is endocytosed upon binding its ligand, VEGF-SLs made to contain encapsulated doxorubicin, are taken up by proliferating vascular endothelial cells, resulting in their destruction. The viability of this method is tested by examining the ability of the liposomes to inhibit tumor growth and/or to eradicate established tumors in vivo. This work provides therefore a novel approach to anti-angiogenic cancer therapy.

### Example 5

The method used to modify the surface of cells by engraftment of recombinant receptors can be used to produce cell-based vaccines that can modify immunological responses when used in vivo.

This is demonstrated by the fact that P815 cells engrafted with the co-stimulator molecules B7.1 and CD40 can be used as a vaccine to enhance tumor immunity. Analogously, these or any other recombinant protein or molecules (possessing the appropriate tag) may be engrafted onto any other biological membranous structure(s) (e.g. a membranous structure derived from cells and/or sub-cellular components, such as plasma membranes vesicles etc.). The engrafted structures can then be used as a vaccine to enhance tumor immunity and/or modify immunological responses in vivo for therapeutic purposes. The preferred method comprises:
(i) incubating the cells or membranous material with a chelator lipid such as NTA-DTDA to allow the lipid to incorporate with the cells or membranes;
(ii) washing off any unincorporated lipid by centrifugation or filtration and resuspension of the structures in the appropriate solution or buffer;
(iii) incubating the membranous structures containing incorporated chelator lipid with an appropriate recombinant protein(s) possessing an appropriate affinity tag; and
(iv) washing off unincorporated protein material; and then using the modified structure as a vaccine administrable in vivo for therapeutic purposes such as to modify immunological responses in vivo.

The subject approach can also be used with synthetic membrane structures (i.e. synthetic liposomes or vesicles composed of a mixture of any phospholipid (e.g. PC or PE) and the NTA-DTDA. The synthetic structures can be made to incorporate the NTA-DTDA, therefore, only steps (iii) to (v) above are required.

### Example 6

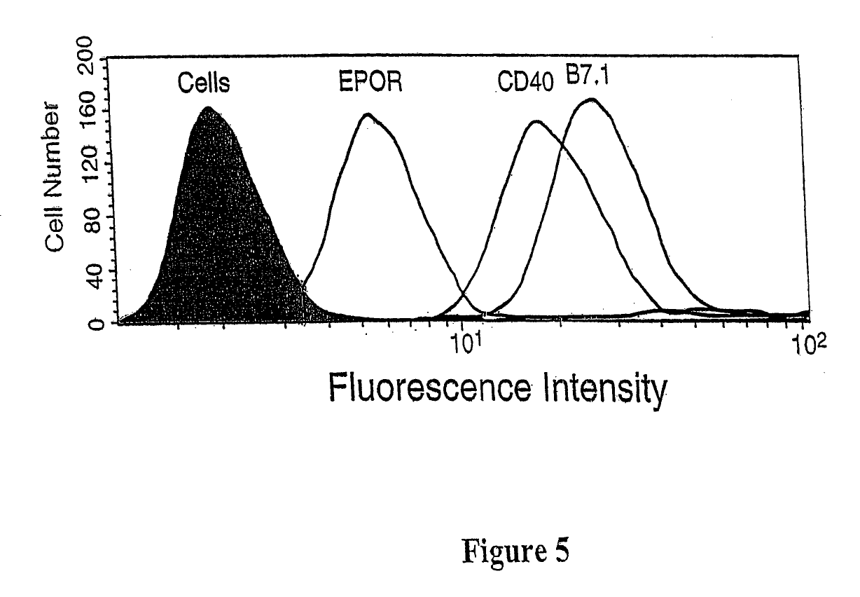

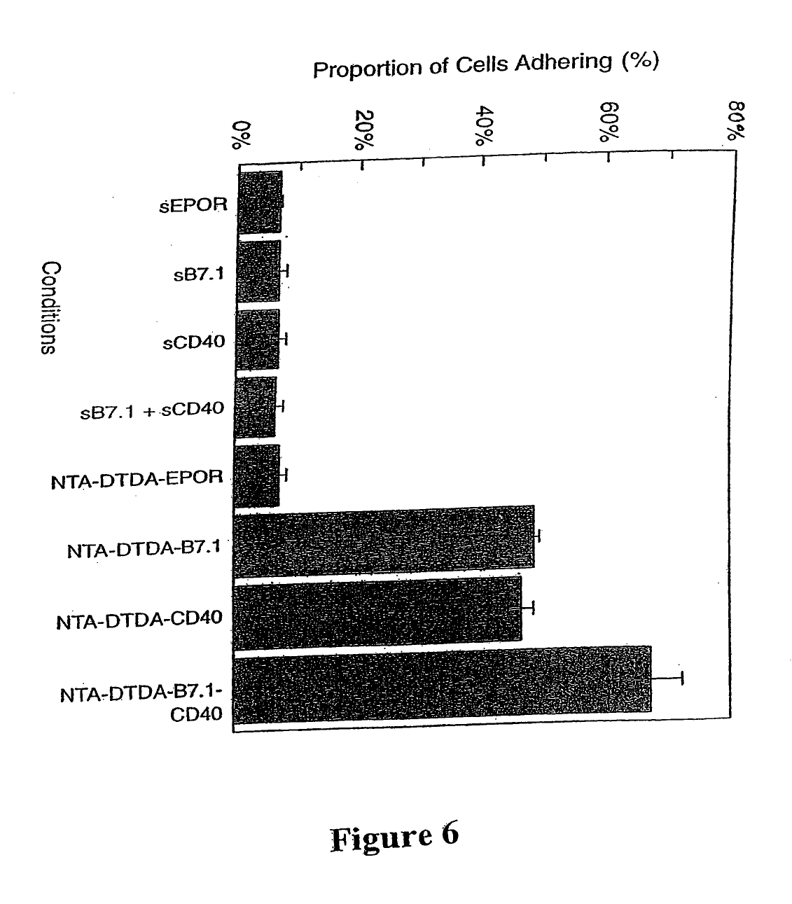

Preliminary experiments indicate that synthetic liposomes (composed of say PC and NTA-DTDA in 10:1 ratio) engrafted with an appropriate recombinant receptor protein can be used to specifically target cells bearing the cognate receptor or ligand (see FIG. 5). Such liposomes also can be used to modify biological response (see FIG. 6). The present invention provides, therefore, a method of modifying the surface of liposomes for use in therapeutic applications to deliver an encapsulated drug or other therapeutic agent to cells or tissues within the body. Such liposomes are used to modify a biological response(s) for the treatment of disease, or for targeting the delivery of cytotoxic drugs or agents to specific cells (e.g. tumor cells) in order to destroy such cells for therapeutic purposes.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

1. Shao, Z. (1995) Quart. Revs Biophys. 28:195-251.

2. Garland, P. B. (1996) Quart. Revs Biophys. 29: 91-117.

3. Khilko et al. (1995) J. mmunol. Meth. 183: 77-94.

4. Velge-Rousel et al. (1995) J. Immunol. Meth. 183: 141-148.

5. Gershon, P. D. and Khilko, S. (1995) J. Immunol. Meth 183: 65-67.

6. Plant et al. (1995) Analyt. Biochem. 226: 342-348.

7. Dietrich et al. (1995) Proc. Nat. Acad. Sci. (USA) 92: 9014-9018.

8. Dietrich et al. (1996) Biochemistry 35: 1100-1105.

9. Du et al. (1997) Biochim Biophys Acta, 1326(2), 236-248.

10. Owens, T. (1996) Current Biology 6: 32-35.

11. Guinan et al. (1994) Blood 84: 3261-3282.

12. Hellstrom et al. (1995) Immunol. Rev. 145: 123-145.

13. La Motte et al. (1996) Cancer Immunol. Immunother. 42: 161-169.

14. Chaux et al. (1996) Int. J. Cancer 66: 244-248.

15. Fujii et al. (1996) Int. J. Cancer 66: 219-224.

16. Martin-Fontecha et al. (1996) Eur. J. Immunol. 26: 1851-1859.

17. Yang et al. (1995) J. Immunol. 154:2794-2800.

18. Shinde et al. (1996) J. Immunol. 157: 2764-2768.

19. Cavallo et al. (1995) Eur. J. Immunol. 25:1154-162.

20. Brunschwig et al. (1995) J. Immunol. 155: 5498-5505.

21. McHugh et. al. (1995) Proc. Nat. Acad. Sci. 92:8059-8063.

22. Schnmitt et al. (1994) J. Am. Chem. Soc. 116:8485-8491.

23. Altin et al. (1994) Eur. J Immunol. 24:450-457.

24. Parish et al. (1974) Eur. J. Immunol. 4:808-815.

25. Greenfield et al. (1997) J. Immunol. 158:2025-2034.

26. Chen et al. (1994) Cancer Res. 54:5420-5423.

27. Achen M. G. and Stacker, S. A. (1988) Int. J. Exp. Pathol. 79:255-265.

28. Maxwell et al. (1997) Proc. Nat. Acad. Sci. (USA) 94:8104-8109.

29. Brown, J. M. and Giaccia, A. J. (1998) Cancer Research 58:1408-1416.

30. Weismann et al. (1997) Cell 91:695-704.

31. Wang et al. (1998) J. Cancer Res. Clin. Oncol. 124:615-620.

32. Lin et al. (1998) Cell Growth Differ. 9:49-58.

33. Papahadjopoulos et al. (1991) Proc. Natl. Acad. Sci. (USA) 88:11460-11464.

34. Allen et al. (1991) Biochim. Biophys. Act. 1066:29-36.

35. Lasic, D. D. and Papahadjopoulos D. (1995) Science 267:1275-1276.

36. Ahmad et al. (1993) Cancer Research 53:1484-1488.

37. Hansen et al. (1995) Biochim. Biophys. Act. 1239:133-144.

38. DeMenezes et al. (1998) Cancer Research 58:3320-3330.

39. Kirpotin et al. (1997) Biochem. 36:66-75.

40. Vaage et al. (1999) Int. J. Cancer. 80:134-137.

What is claimed is:

1. An in vitro method of anchoring a molecule directly onto biological membranes, said method comprising:
(i) preparing a suspension of a chelator lipid or liposomes containing the chelator lipid;
(ii) incubating a suspension of cells or biological membranous structures with a suspension of the chelator lipid to allow the chelator lipid to incorporate into the structures;
(iii) washing away excess or unincorporated lipid;
(iv) incubating the membranous structures with a solution of the molecule to be anchored, said molecule possessing an appropriate metal affinity tag; and
(v) washing away excess or unbound soluble protein, and suspending the structures in a solution suitable for administration in vivo.

2. A method as claimed in claim 1 wherein said molecule to be anchored has a binding partner on a particular cell or tissue to allow targeting of said cells or biological membranes to which the molecule is anchored to the particular cells or tissue.

3. A method according to claim 1 wherein the molecule is a ligand, receptor, recombinant protein, polysaccharide, glycoprotein, nucleic acid molecule or antigen.

4. A method according to claim 1 wherein the molecule is a co- stimulatory molecule.

5. A method according to claim 1 wherein the anchored molecule is VEGF.

6. A method according to claim 4 wherein the co-stimulatory molecule is CD40 or B7.1.

7. A method according to claim 1 wherein the chelator lipid comprises a metal chelating group.

8. A method according to claim 7 wherein the metal chelating group is nitrilotriacetic (NTA).

9. A method according to claim 1 wherein the molecule to be anchored comprises a metal binding polypeptide tag.

10. A method according to claim 9 wherein the polypeptide tag comprises a sequence of at least six amino acid residues.

11. A method according to claim 10 wherein the polypeptide tag comprises hexa-histidine.

12. A method according to claim 1 wherein the biological membrane is from a tumor cell.

13. A method of targeting a membranous structure to a particular cell or tissue within a subject, wherein the membranous structure comprises cells or biological and/or synthetic membranes or liposomes, said method comprising engrafting to said membranous structure a molecule having a binding partner on the particular cell type or tissue to be targeted by:
(i) preparing a suspension of a chelator lipid or liposomes containing the chelator lipid;
(ii) incubating a suspension of membranous structures with a suspension of the chelator lipid to allow the chelator lipid to incorporate into the structures;
(iii) washing away excess or unincorporated lipid;
(iv) incubating the membranous structures with a solution of the molecule to be anchored, said molecule possessing an appropriate metal affinity tag; and
(v) washing away excess or unbound soluble protein, and suspending the structures in a solution suitable for administration in vivo;
and administering the membranous structure to the subject.

14. A method according to claim 13 wherein the molecule is a ligand, receptor, recombinant protein, polysaccharide, glycoprotein, nucleic acid molecule or antigen.

15. A method according to claim 13 wherein the anchored molecule is VEGF.

16. A method according to claim 13 wherein the chelator lipid comprises a metal chelating group.

17. A method according to claim 16 wherein the metal chelating group is nitrilotriacetic (NTA).

18. A method according to claim 13 wherein the molecule to be anchored comprises a metal binding polypeptide tag.

19. A method according to claim 18 wherein the polypeptide tag comprises a sequence of at least six amino acid residues.

20. A method according to claim 19 wherein the polypeptide tag comprises hexa-histidine.

21. A method according to claim 13 wherein the biological membrane is from a tumor cell.
